(19) United States
(12) Patent Application Publication
(10) Pub. No.: US 2020/0206134 A1
(43) Pub. Date: Jul. 2, 2020

(54) POLYETHYLENIMINE NANOPARTICLES AND METHODS OF USING SAME

(71) Applicants: Children's Hospital Medical Center, Cincinnati, OH (US); University of Cincinnati, Cincinnati, OH (US)

(72) Inventors: Vladimir Kalinichenko, Cincinnati, OH (US); Andrew Dunn, Worthington, OH (US); Donglu Shi, Loveland, OH (US)

(21) Appl. No.: 16/623,448

(22) PCT Filed: Jul. 17, 2018
(86) PCT No.: PCT/US18/42362

§ 371 Date: Dec. 17, 2019

Related U.S. Application Data
(60) Provisional application No. 62/533,238, filed on Jul. 17, 2017.

(51) Int. Cl. A61K 9/107; A61K 47/59; A61K 47/54; A61K 9/00; A61K 38/17

(52) U.S. Cl. CPC A61K 9/107; A61K 47/59; A61K 38/17; A61K 9/0019; A61K 47/543

(57) ABSTRACT

Disclosed herein are nanoparticle compositions containing that may be created by functionalizing polyethylenimine (PEI) with fatty acids and carboxylate terminated poly(ethylene glycol) (PEG). The disclosed compositions may be delivered to an individual in need thereof via delivery into blood circulation, where the nanoparticle compositions show an exceptionally high specificity to the pulmonary microvascular endothelium with minimal targeting of other cell types in the lung, to provide delivery of therapeutic agents such as stabilized nucleic acids. Methods of using the compositions are also disclosed.

### CROSS-REFERENCE TO RELATED APPLICATIONS

This application claims priority to and benefit of U.S. Provisional Application Ser. No. 62/533,238, filed Jul. 17, 2017, the contents of which are incorporated in its entirety for all purposes.

### BACKGROUND

Pulmonary vascular disease (PVD) encompasses a wide range of pediatric and adult pulmonary disorders, such as pulmonary hypertension, alveolar capillary dysplasia, and various arterial, venous, and lymphatic malformations. PVD is associated with poor prognosis in patients with bronchopulmonary dysplasia, a severe respiratory disorder of infants. Gene therapy by adenovirus vectors has shown to ameliorate pulmonary hypertension, and stimulate endothelial repair after chronic lung injury. However, major detractions of viral vectors to clinical translation are their random integration into the genome and potent ability to antagonize a significant immune response. Efficient, non-viral delivery systems specifically targeting the pulmonary endothelium are therefore critically needed to treat PVD. The instant invention addresses one or more of the aforementioned needs in the art.

### BRIEF SUMMARY

Disclosed herein are nanoparticle compositions containing that may be created by functionalizing polyethylenimine (PEI) with fatty acids and carboxylate terminated poly(ethylene glycol) (PEG). The disclosed compositions may be delivered to an individual in need thereof via delivery into blood circulation, where the nanoparticle compositions show an exceptionally high specificity to the pulmonary microvascular endothelium with minimal targeting of other cell types in the lung, to provide delivery of therapeutic agents such as stabilized nucleic acids. Methods of using the compositions are also disclosed.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1A-1D FIG. 1A) atr-FTIR spectrum of myristic acid (dotted), PEI600-MA5 (dashed), and PEI10k-LinA15-PEG3.0 (solid) showing amidation after conjugation as well as inclusion of PEG and linoleic acid on to PEI10k. FIG. 1B) 1H NMR spectrum of conjugated polymers. FIG. 1C) Gel electrophoresis analysis of CMV-empty plasmids bound to conjugated PEI at varying mass ratios of polymer:DNA (w/w). FIG. 1D) Hydrodynamic size distribution of PEI10k-LinA15-PEG3.0 in normal glucose used for I.V. injection.

FIGS. 2A-2C. FIG. 2A) Gating strategy for identification of lineage populations from live singlet cells isolated from whole lung. Population (a) is identified as the hematopoietic population, (b) as the endothelial population, (c) as the epithelial population, and (d) as the lineage negative population. FIG. 2B) Histogram analysis of PEI10k-LinA15-PEG3.0 (blue curve) targeting against the fluorescence minus one control (red curve). Numeric values represent the average±σ (n=3). FIG. 2C) Juxtaposition of lineage targeting from three novel formulations. PEI1800-LinA5-PEG0.3 significantly increased endothelial targeting. PEI₁₀ₖ-LinA₁₅-PEG_{3.0} significantly increased endothelial and decreased epithelial targeting compared to PEI600-MA5/PEG-OA/Cho. Inset) Median fluorescent intensity (MFI) analysis of isolated endothelial populations from eGFP RNA transfected mice compared to fluorescent minus one (FMO) control without injected eGFP RNA (n=5). *(p<0.05), ***(p<0.001).

FIGS. 3A-3C. Immunofluorescence of frozen lung sections post I.V. injection of labeled PEI₁₀ₖ-LinA₁₅-PEG_{3.0}. FIG. 3A) Microvasculature shows nanoparticles disseminated throughout PECAM1 expressing cells (panels a, b). FIG. 3B) Large vessels, identified by αSMA staining, are associated with reduced presence of nanoparticles (panels c, d). FIG. 3C) Nanoparticles along PECAM1 cells within the lumen of large vessels (panels e, f).

FIGS. 4A-4D. FIG. 4A) 3D deconvolution of PECAM1 (green) cells within the microvasculature showing colocalization with labeled PEI₁₀ₖ-LinA₁₅-PEG_{3.0} nanoparticles (red). FIG. 4B) Surface reconstruction from a maximum intensity projection shows nanoparticle fluorescence with subcellular and surface localization. FIG. 4C) Percent internalization of nanoparticle fluorescence within PECAM1 cells calculated from the 3D deconvolution. FIG. 4D) IVIS live in-vivo imaging of labeled PEI₁₀ₖ-LinA₁₅-PEG_{3.0} nanoparticles following I.V. injection at FIG. 4D, panel a) 24 hours, FIG. 4D, panel b) 72 hours, and FIG. 4D, panel c) 7 days. Maximum fluorescence is found to be localized near the lungs and kidneys.

FIG. 5. High targeting percentages within the gated live endothelial population (CD31+ CD45− CD326−) are observed for a multitude of major organs. Lung shows the highest targeting percentage with ˜80% for the PEI₆₀₀-MA₅/PEG2k-OA/Cho (100:11.1:11.1) formulation followed closely by liver, kidney, spleen, and heart. All examined organs show at least 50% targeting within the live endothelial population.

FIG. 6. Schematic showing (1) Activation of carboxylate group. (2) Amidation following PEI addition. (3) Purification by dialysis and extraction.

FIG. 7. atr-FTIR of PEI1800-LinA5-PEG_{0.3} showing alkene inclusion from linoleic acid and ether bonding from PEG conjugation.

FIG. 8. Full gating strategy for nanoparticle targeting analysis showing singlet isolation.

FIGS. 9A-9C. Targeting dependence of nanoparticles (blue) on formulation relative to FMO control (red), 2 hours post I.V. injection. FIG. 9A) PEI1800-OA₈-MA₂-PEG_{5k, 1.0}, FIG. 9B) PEI₆₀₀-OA_{3.25}-SA_{0.75}, FIG. 9C) PEI₆₀₀-OA_{1.5.}

FIG. 10. 10× immunofluorescence of frozen lung sections post I.V. injection of DyLight 650 labeled PEI₁₀ₖ-LinA₁-PEG_{3.0}. Sections were stained with Hoechst 33342 (nuclear stain), platelet endothelial cell adhesion molecule (PECAM1, CD31), and alpha smooth muscle actin (αSMA) for visualization of microvasculature and large vessels.

FIGS. 11A-11F. Decreased endothelial cell proliferation and STAT3 signaling in S52F-Foxf1+/− mice. (FIG. 11A) PECAM1 and FLK1 staining was decreased in lungs of E15.5 S52F-Foxf1+/− embryos. (FIG. 11B) Protein and mRNA of Flk1 and Pecam1 were reduced in lungs from E15.5 S52F-Foxf1+/− mice as shown by Western blot (upper panel) and qRT-PCR (bottom panel). (FIG. 11C) Decreased pulmonary endothelial cell proliferation in the S52F-Foxf1+/− mice is shown using Ki-67 and BrdU immunostaining. (FIG. 11D) Graphical representation of cell proliferation by Ki-67 and BrdU staining. Percentage of Ki-67-positive and BrdU-positive cells was counted in ten random microscope fields (n=3 mice in each group). (FIG. 11E FIG. 11F) Immunoblots and qRT-PCR data show decreased total STAT3 and phospho-STAT3 (Tyr705) in lungs of S52F-Foxf1+/− and Foxf1+/− E18.5 embryos. mRNA was normalized to β-actin mRNA. *indicates p<0.05.

FIGS. 12A-12G. Nanoparticle-mediated delivery of STAT3 restores endothelial cell proliferation and angiogenesis in S52F-Foxf1+/− newborn mice. (FIG. 12A) FACS gating strategy for the (a) hematopoietic, (b) endothelial, (c) epithelial, and (d) lineage negative cells with histograms highlighting respective cell selective targeting (n=3 mice). (FIGS. 12B-12C) Immunoblots show the levels of STAT3, pSTAT3, FLK-1, PECAM-1, and PDGFb in lung extracts after nanoparticle-mediated delivery of CMV-STAT3 via facial vein. CMV-empty was used as a control. Nanoparticle/DNA complexes were injected at P2 and mice were harvested at P7. Images were quantified using densitometry (n=3 mice). p<0.05 is *. (FIG. 12D) qRT-PCR shows the expression of Flk1 and Pecam1 mRNAs in P7 lungs after nanoparticle mediated delivery of CMV-STAT3. (FIG. 12E) Images show the Ki-67 (arrowheads) and isolectin B4 (IB4) staining of P7 lungs after nanoparticle mediated delivery of STAT3. (FIG. 12F) Percentage of Ki-67 positive endothelial cells was calculated using 10 random images from 3 mouse lungs in each group. p<0.01 is **. (FIG. 12G) Schematic diagram shows the proposed molecular mechanisms whereby FOXF1 regulates STAT3 signaling.

FIG. 13. Accumulation of DyLight 650-conjugated PEI₆₀₀-MA5.0 nanoparticles in FACS-sorted cells. Bar graph shows mean fluorescence intensity of DyLight 650 in different cell populations of WT lungs harvested 24 hr after injections of nanoparticles. Statistical significance (<0.05) was calculated using an unpaired t-test assuming unequal variance (n=3 mice).

FIGS. 14A-14C. EDC/NHS based conjugation scheme. (FIG. 14A) PEI₆₀₀-MA5.0 atr-FTIR showing amide carbonyl stretching v=1650 cm−1 and the disappearance of carboxylic acid stretching v=1290 cm−1 in the conjugated polymer. (FIG. 14B) FACS gating strategy for identification of hematopoietic (a), endothelial (b), lineage negative (c) and epithelial (d) cells in lung tissue. (FIG. 14C) Polyplex size and zeta potentials reported from DLS measurements in normal glucose at a w/w ratio of 24. Respective distribution of colloidal sizes from DLS.

FIG. 15. Nanoparticle delivery of CMV-STAT3 inhibits lung inflammation in S52F-Foxf1+/− lungs. Nanoparticles/DNA complexes were injected at P2, lungs were harvested at P7. CMV-STAT3 reduces lung inflammation and improves lung structure in S52F-Foxf1+/− neonates.

FIG. 16. FOXF1 stimulates STAT3 transcription Immunohistochemical staining of human ACDMPV lung sections shoes decreased pSTAT3, Ki-67, FLK1 and Cyclen D1 (n=3 in each group).

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

Pulmonary vascular disease encompasses a wide range of serious afflictions with important clinical implications. There is a critical need for the development of targeted, efficient, non-viral gene therapy delivery systems for tailored treatment to reduce potentially dangerous off-target effects. Disclosed herein are methods and compositions that provide cell targeting via a uniquely designed nanosystem. The disclosed novel formulations of cationic based, non-viral nanoparticles may be used to enable efficient targeting of tissues, for example, the pulmonary microvascular network, for the delivery of particles such as nucleic acids.

Applicant has found that the nanoparticles disclosed herein may be created by functionalizing low and medium molecular weight polyethylenimine (PEI) with biological fatty acids and carboxylate terminated poly(ethylene glycol) (PEG) through a one-pot EDC/NHS reaction. After delivery into blood circulation, the nanoparticles show an exceptionally high specificity to the pulmonary microvascular endothelium with minimal targeting of other cell types in the lung. Thus, the described nanoparticles may be used for the successful delivery of stabilized nucleic acids such as RNA. Live in-vivo imaging, flow cytometry of single cell suspensions, and confocal microscopy were used to demonstrate that polyplexes are enriched in the lung tissue and disseminated in 91.3±1.8% of alveolar capillary endothelium while sparse in large vessels. Thus, these polyplexes therefore may be used to provide a powerful basis for targeted, disseminated delivery of nucleic acids to the pulmonary microvasculature.

In one aspect, disclosed herein are micelle compositions for delivery of a therapeutic agent. The composition may comprise a polyethylenimine (PEI) conjugated to a fatty acid (FA) to form a PEI-FA conjugate. The PEI-FA conjugate may then aggregate to form a micelle, for example, a cationic micelle.

In one aspect, the PEI-FA conjugate may further be conjugated to a carboxylate-terminated polyethylene glycol (PEG) to form a PEI-FA-PEG conjugate, wherein said PEI-FA-PEG conjugate may aggregate to form a micelle, for example, a cationic micelle.

In one aspect, the PEI used for the disclosed compositions may have an Mn (number average molecular weight) of from about 600 Da to about 10 kDa, or about 1000 Da to about 2500 Da, or about 1200 Da to about 1800 Da, wherein Mn is defined as (when n=1):

$$\frac{{\sum }_{i = 1}^{p} {Mn}_{i}^{n} * {N}_{i}}{{\sum }_{i = 1}^{p} {Mn}_{i}^{n - 1} * {N}_{i}}$$

where the molecular weight distribution is quantized into (p) fractions, (Ni) and (Mnᵢ) are the number of molecules in the i^{th} fraction and molecular weight in the i^{th} fraction respectively. In one aspect, the polyethylenimine (PEI) may be a branched polyethylenimine (PEI), which may contain primary, secondary and tertiary amino groups.

In one aspect, the PEI-FA ratio may be from about 3 to about 30, or wherein said PEG-FA ratio is from about 1 to about 2. In one aspect, the micelle may have a molar conjugation ratio (grafting density) of about 3 to about 5 moles of fatty acids per mole of PEI₆₀₀. In one aspect, the micelle may have a molar conjugation ratio (grafting density) of about 3 to about 8 moles of fatty acids per mole of PEI₁₈₀₀. In one aspect, the micelle may have a molar conjugation ratio (grafting density) of about 3 to about 10 to about 30 moles of fatty acids per mole of PEI₁₀ₖ. As used herein, “grafting density” is the molar degree of conjugation (moles of fatty acids per mole of PEI). This may refer to the total molar number of fatty acids or, in the case of a mixture of fatty acids, the molar number of each individual fatty acid type.

In one aspect, where PEG is conjugated to the PEI-FA conjugate, the PEG may have an Mn (number average molecular weight) of from about 2 kDa to about 5 kDa. In one aspect, the PEI may have an Mn (number average molecular weight) of about 600 Da to about 10 kDa.

In one aspect, the micelle may have a size of from about 80 nm to about 200 nm, or about 100 nm to about 150 nm as quantified by Dynamic Light Scattering (DLS). In one aspect, the micelle may have a Zeta (Surface) Potential of from about 5 mV to about 34 mV, or about 20 mV to about 30 mV as quantified by Dynamic Light Scattering (DLS).

In one aspect, the fatty acid may be a biological fatty acid. For example, the fatty acid may be selected from any saturated or unsaturated fatty acid with a tail length of 12-16 carbons, for example, including, but not limited to, lauric acid, myristic acid, palmitic acid, myristoleic acid, palmitoleic acid, sapienic acid, oleic acid, linoleic acid, a-linolenic acid, or combinations thereof.

In one aspect, the micelle may further comprise cholesterol at a mass percentage of up to about 15% relative to all polymeric and conjugated polymeric components comprising the nanoparticle, wherein the cholesterol may be present in an amount sufficient to improve colloidal stability. In one aspect, cholesterol may be included to reduce colloid size when conjugated PEI colloids are greater than 200 nm in hydrodynamic diameter as quantified by DLS.

In one aspect, the disclosed compositions may further comprise a therapeutic agent. The therapeutic agent may be selected from a hydrophobic peptide, a hydrophobic small molecule, or a nucleic acid. The micelle may be used to incorporate or encapsulate the therapeutic agent for delivery to an individual in need thereof.

In one aspect, the therapeutic agent may be a nucleic acid selected from DNA and RNA. In one aspect, the nucleic acid may be in the form of a non-integrating, self-replicating plasmid (Enhanced Episomal Vector). The therapeutic agent may be, in certain aspects, a nucleic acid selected from a pro-angiogenic or anti-angiogenic gene, for example, STAT3 (Signal Transducer and Activator of Transcription 3), FoxF1 (Forkhead Box F1 transcription factor), or a combination thereof. Other genes may include any FoxF1 or STAT3 target genes.

In one aspect, the composition may be in the form of a micelle and have a zeta potential of from about 5 to about 35 mV, or about 20 to about 30 mV. For zeta potential measurements and solution pH, a buffer strength of 10-25 mM may be added. In one aspect, a MOPS buffer may be used.

In one aspect, the composition may be provided in a solution having a pH of between about 7.3 to about 7.5 as measured by electrochemical potential.

In one aspect, the composition may be provided in normal glucose buffered to physiological pH.

In one aspect, the composition may comprise glucose or trehalose in an amount sufficient to serve as a cryoprotectant for the freeze-drying of samples for long term storage.

### Methods of Using

In one aspect, a method of targeting a therapeutic agent to an individual having an endothelial-based disease is disclosed. The endothelial-based disease may be a vascular disease/abnormality, or a pulmonary vascular disease (PVD). In one aspect, the PVD may be selected from pulmonary arterial hypertension, vascular neoplasm, alveolar capillary dysplasia, arterial malformation, venous malformation, lymphatic malformation, bronchopulmonary dysplasia, pulmonary fibrosis, cystic obstructive pulmonary disease (COPD), interstitial lung disease, emphysema, and any cancers where tumor vasculature is the intended target, or combinations thereof. The method may comprise the step of intravenous administration to the individual. The method may comprise the step of administering any composition as described above, to an individual in need of such treatment, particularly wherein the disclosed composition may comprise a nucleic acid.

The administration step may also include inhalation by intratracheal instillation, in particular for epithelial targeting. The administration step may also be selected from intravenous, subcutaneous, oral, or parenteral. In some embodiments, compositions provided herein may be formulated into liquid preparations such as suspensions, syrups, elixirs, and the like. Unit dosage forms may be configured for administration for a pre-determined dosage regimen, for example, a unit dosage form for administration once a day, twice a day, or more.

In one aspect, pharmaceutical compositions may be isotonic with the blood or other body fluid of the recipient. The isotonicity of the compositions may be attained using sodium tartrate, propylene glycol or other inorganic or organic solutes.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected.

In one aspect, the disclosed compositions may be provided in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used.

In some embodiments, the active agents provided herein may be provided to an administering physician or other health care professional in the form of a kit. The kit is a package which houses a container which contains the disclosed composition, and instructions for administering the composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Pulmonary vascular disease (PVD) encompasses a wide range of pediatric and adult pulmonary disorders, such as pulmonary hypertension, alveolar capillary dysplasia, and various arterial, venous, and lymphatic malformations.^{[1-4]} PVD is associated with poor prognosis in patients with bronchopulmonary dysplasia, a severe respiratory disorder of infants. ^{[5-7]} Gene therapy by adenovirus vectors has shown to ameliorate pulmonary hypertension and stimulate endothelial repair after chronic lung injury. ^{[8, 9]} However, major detractions of viral vectors to clinical translation are their random integration into the genome and potent ability to antagonize a significant immune response. ^{[10-14]} Efficient, non-viral delivery systems specifically targeting the pulmonary endothelium are therefore critically needed to treat PVD.

Polyethylenimine (PEI) has been used successfully for non-viral transfection with higher molecular weight. High molecular weight, branched PEI has been shown to be more efficient than low molecular weight PEI and more resistant to aggregation in salt solutions than linear PEI.^{[15-17]} A drawback of higher molecular weight PEI is the substantial increase in toxicity in vitro and in vivo juxtaposed with low molecular weight PEI.^{[17]} Recent research has seen the modification of low molecular weight PEI for reduced toxicity and improved transfection efficiency.^{[18]} Modification of PEI has been done through ring opening synthesis,^{[19-21]} amidation by activated carboxylate groups,^{[22-24]} through the Schotten-Baumann reaction using carboxylic acid chlorides, ^{[25]} and by Micheal Addition. ^{[26-28]} The grafting of small alkane tails, aryl, and hydrophobic groups induces amphiphilic behavior, allowing for the formation of nano-colloids in solution.^{[19, 26, 29-31]} Further inclusion of poly(ethylene glycol) reduces serum binding and opsionization, increasing circulation time.^{[32-35]} This modification essentially creates a pseudo-lipid which spontaneously forms micellar structures in aqueous solutions.

Colloidal stability of these lipid-like micelles can be improved through the inclusion of cholesterol, with an observed decrease in colloidal size. ^{[20]} Incorporation of PEG into the micelle follows a similar approach to cholesterol in which PEG2k has been conjugated to hydrophobic alkane tails and incorporated through microfluidic mixing. ^{[20]} Polymeric based gene delivery research has commonly focused on local injections to a target region. This delivery strategy is not widely applicable for translational application, especially in the case of large target areas requiring widespread dissemination. Here Applicant has developed low and medium molecular weight PEI based nanoparticles capable of targeting the pulmonary endothelium with exceptionally high efficiency for the delivery of nucleic acids.

### 2. Materials and Methods

2.1 Materials: Methoxypolyethylene glycol amine Mn=2000 (PEGNH₂) was obtained through Nanocs. O-Methyl-O′-succinylpolyethylene glycol Mn=2000, Polyethylenimine, Mn=600, 1800, 10k (PEI₆₀₀, PEI₁₈₀₀, PEI₁₀ₖ), Myristic Acid ≥99%, Linoleic Acid (LinA) ≥99.0%, Oleic Acid (OA ≥99%), Myristic Acid (MA ≥99%), Cholesterol (BioReagent ≥99%), Ethanol (EtOH, 200p), HPLC grade water, 2-(N-morpholino)ethanesulfonic acid (MES) ≥99%, 3-(N-Morpholino)propanesulfonic acid (MOPS) were obtained through Sigma-Aldrich and used without further purification. 1-ethyl-3-(3-dimethylaminopropyl)carbodiimide hydrochloride (EDC), N-hydroxysuccinimide (NHS), and DyLight 650 NHS Ester were obtained through ThermoFisher Scientific and used as received. Spectrum™ Spectra/Por™ 3.5 kDa and 20 kDa MWCO dialysis tubing were obtained through Fisher Scientific. Hoechst 33342 and ProLong™ Diamond was purchased from ThermoFisher. Stabilized eGFP RNA was obtained as a generous gift from TranscripTX.

autoMACS running buffer was obtained from Miltenyl Biotec. Fixable Viability Dye eF780 was obtained from eBioscience. Dulbecco's Modified Eagle's Medium, L-glutamine (100×), and antibiotic-antimycotic (100×) were obtained through ThermoFisher Scientific.

Antibodies (Ab): anti-mouse CD16/CD32 (eBioscience, clone 93), anti-mouse CD31-eF405 (eBioscience, clone 390), anti-mouse CD45-eVolve655 (eBioscience, clone 30-F11), anti-mouse CD326-PerCP-eF710 (eBioscience, clone G8.8). Rat anti-mouse CD31 (BD Bioscience, clone MEC13.3), Mouse anti-mouse αSMA, Donkey anti-rat-AlexaFlour488 (ThermoFisher), Donkey anti-mouse-AlexaFluor594 (ThermoFisher)

Buffers: MES was dissolved into double distilled H₂O to a concentration of 500 mM. pH was adjusted to 6.0 with 5 N NaOH. MOPS was dissolved into double distilled H₂O to a concentration of 100 mM. The pH was adjusted to 7.4 with 2 N NaOH and the buffer diluted to 10 mM. Buffer solutions were then filtered through a 0.22 μm filter.

2.2 Conjugated Polyethylenimine: Functionalization of PEI with biological fatty acids and PEG was completed through amidation using EDC/NHS mediated coupling. A general reaction scheme was used for all coupling reactions. For PEI conjugation, the mass of EDC was based on the EDC:COOH molar ratio of 1.25:1 and the mass of NHS was based on the NHS:EDC molar ratio of 1.25:1. EDC:COOH and NHS:COOH ratios for PEGNH2 conjugation were 1.25:1 and 2:1 respectively. 500 mM MES buffer volume, pH=6, was based upon the molar ratio of 30:1, H₂O:COOH. Initially, EDC and NHS were solvated in EtOH with half the volume of MES buffer and allowed to react for 15 minutes. A predetermined amount of PEI was solvated in EtOH with the remaining volume of MES buffer. The total volume of EtOH was determined to be the volume required for a final concentration of 95% EtOH. A final concentration of 99% EtOH was used for PEGNH₂ conjugation. Solvated PEI was quickly added following carboxylate activation and the solution was allowed to react overnight at 40° C. EtOH was removed by rotary evaporation following conjugation and the resulting product was resuspended in deionized H₂O. Conjugated PEI was dialyzed against deionized H₂O using a 20 kDa membrane for 4-5 days, extracted twice in diethyl ether, and lyophilized Lyophilized polymers were suspended in 10 mM MOPS, pH=7.4 and sonicated prior to use using a cup horn sonicator. Cholesterol (Cho) was solvated into EtOH at a concentration of 10 mg/ml. Cho and PEG-OA were incorporated into PEI-MA5 colloids through solvent diffusion and microfluidic mixing. Ethanol was removed by dialysis using a 3.5 kDa Slide-A-Lyzer overnight. Polymers were fluorescently tagged using NHS-functionalized fluorophores at a ratio of 12.5 μg of NHS-functionalized fluorophore to 1 mg of polymer in 10 mM MOPS buffer, pH=7.4, and allowed to react overnight at room temperature in the dark.

2.3 Gel Electrophoresis: TBE based agarose gels (0.8% w/v, 0.5× TBE) were used to examine the complexation ratios of DNA with PEI based vectors. CMV-plasmid DNA (1 μg) was incubated at varying mass ratios with PEI based vectors. Complexation was allowed for 15 minutes before gel loading. Gels were run at 120 V and imaged on a Bio-Rad Gel Doc™.

2.4 Polyplex Formation: For sizing and zeta-potential analysis, 10 μg of CMV-plasmid DNA was mixed with polymer formulations at various mass ratios in 100 μl normal glucose supplemented with 10 mM MOPS, pH=7.4, at room temperature. Polyplexes were allowed to rest at room temperature at least 10 minutes before analysis. The surface potential of formulated cationic polyplexes was switched through coating with either poly(acrylic acid) (PAA) or heparin by charge association following this 10 minute period. 20 mg/ml stock solutions of PAA or heparin, buffered to 7.4, were quickly mixed with formulated polyplexes at set mass ratios relative to that of the cationic polymer and allowed to bind for at least 10 minutes before use. For in vivo delivery, 40 μg of CMV driven plasmids were mixed with PEI₆₀₀-MA5/PEG-OA/Cho, PEI₁₈₀₀-LinA5-PEG_{0.3}, and PEI₁₀ₖ-Lin_{A15}-PEG_{3.0} at mass ratios of 21, 25, 15 w/w respectively in normal glucose. These mass ratios correspond to 3×, 10×, and 10× the w/w ratio required to stabilize DNA as determined by gel electrophoresis.

2.5 In Vivo Flow Cytometry: All animal experiments were carried out in accordance to applicable guidelines using approved animal protocols. Mice were given free access to food and water over the course of the study. 40 μg of CMV driven plasmids were mixed with PEI₆₀₀-MA5/PEG-OA/Cho, PEI₁₈₀₀-LinA5-PEG_{0.3}, and PEI₁₀ₖ-Lin_{A15}-PEG_{3.0} at mass ratios of 21, 25, 15 w/w respectively in normal glucose. These mass ratios correspond to 3×, 10×, and 10× the w/w ratio required to stabilize DNA as determined by gel electrophoresis. For stabilized RNA injections, 30 μg of eGFP RNA was mixed with PEI₁₀ₖ-Lin_{A15}-PEG_{3.0} at a mass ratio of 4.5. A final volume of 250 μl or 200 μl was used for tail vein injection of plasmids or RNA respectively into wild type C57BL/6, 8-10 weeks of age. Whole lungs were harvested 24 hours post I.V. injection. FACS analysis was performed using a BD Biosciences LSR II.

Lungs were digested using a lysis buffer of DMEM supplemented with L-glutamine, anti-biotics/mycotics, 0.5 mg/ml DNase, 100 μg/ml liberase. Cells were isolated from the extracellular matrix and blocked in MACS buffer with CD16/CD32 Abs. Cells were then stained with CD31 Ab labeled with eF40, CD45 Ab labeled with eVolve655, and CD326 Ab labeled with PerCP-eF710. Dead cells were stained with fixable viability dye-eF780 (FVD). Populations were gated on live singlets as CD31+ CD45− CD326− (endothelial), CD45+ CD31− CD326− (hematopoietic), CD326+ CD31− CD45− (epithelial), CD45− CD31− CD326− (lineage negative).

2.6 Live Imaging: 40 μg of CMV driven plasmids were mixed with PEI₁₀ₖ-Lin_{A15}-PEG_{3.0} at a mass ratio of 15 w/w respectively in normal glucose. A final volume of 250 μl was used for tail vein injection into adult, nude mice. Mice were anesthetized under 3-5% isoflurane and maintained at 1-2% while imaging. Fluorescence was imaged using standard transillumination.

2.7 Immunofluorescence: Lungs from wild type C57BL6/J mice (8-10 weeks old) were inflated with 1:1 PBS:optimal cutting temperature (OCT) compound and frozen in OCT. 10 μm sections were fixed for 10 minutes at −20° C. in 1:1 methanol:acetone, washed in 0.3% Tween 20 in PBS and blocked in 4% donkey serum/2% BSA/0.1% Tween 20 in PBS. The antibody buffer used during staining was 0.4% donkey serum/0.2% BSA/0.1% Tween 20 in PBST. Rat anti-CD31 and Mouse anti-αSMA were diluted in buffer at 1:250 and 1:2000 dilutions respectively and incubated overnight at 4° C. Slides were washed and incubated with donkey anti-Rat labeled with AF488 and donkey anti-Mouse labeled with AF594 overnight at 4° C. Slides were washed, stained with Hoechst 33342, and mounted with ProLong™ Diamond on #1.5 coverglass. Imaging was done using a Nikon A1 confocal microscope with Richardson-Lucy deconvolution in Nikon Elements and analysis performed in Imaris.

2.8 Characterization: Infrared spectroscopy was run on a Nicolet attenuated total reflection Fourier transform infrared (atr-FTIR) spectrometer outfitted with a diamond crystal. NMR was taken in deuterated chloroform on a Bruker AV 400 MHz spectrometer. Hydrodynamic size and zeta potential were measured on a Malvern Zetasizer Nano ZS in normal glucose.

Degree of Conjugation (DoC): Fatty acid and PEG conjugation onto PEI was calculated through 1H NMR spectroscopy using the terminal methyl group of the conjugated fatty acid (a), the integrated peak from the PEI backbone (g), and the integrated peak from PEG (c). Myristic acid and linoleic acid gave rise to ¹NMR peaks that overlap with the PEI spectrum in (g). Therefore, the following calculation method was used to decouple the two signals where (P) is the relative integration of the PEI+fatty acid peak, (Z) is the relative integration of the terminal methyl peak, (B) is the number of hydrogens contributing to (P) relative to the terminal methyl group. For myristic and linoleic acid, B is equal to 2 and 4 respectively. (X) is the decoupled, relative PEI integration. (Y) is the decoupled, relative fatty acid integration, and (C) is the total number of hydrogens in the PEI backbone as estimated from molecular weight. For PEG conjugation, only Eqs. 1 and 3 were used; (Y) and (B) in Eq. 3 were then equivalent to the relative PEG integration and the total number of hydrogens in the PEG backbone determined from molecular weight.

$$\begin{matrix}[ P - Z * ( B / 3 ) ] = X & ( 1 ) \\ P - X = Y & ( 2 ) \\ \frac{C - DoC}{X} * \frac{Y}{B} = DoC & ( 3 )\end{matrix}$$

### 3. Results

3.1 Synthesis and Characterization: A schematic diagram of the synthesis method is shown in as follows, wherein (1) illustrates activation of carboxylate group, (2) illustrates amidation following PEI addition, and (3) illustrates purification by dialysis and extraction.

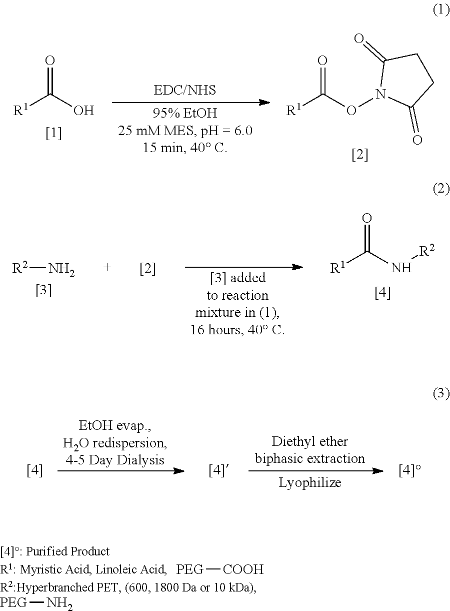

This scheme was further used for the functionalization of oleic acid to 2 kDa carboxylate terminated PEG (PEG-OA). 600 Da PEI (PEI₆₀₀) was functionalized with myristic acid (MA) in a 1:5 molar ratio (PEI₆₀₀-MA5). Linoleic acid (LinA) and 2 kDa PEG was conjugated to 1.8 kDa and 10 kDa PEI in 1:5:0.3 and 1:15:3 molar ratios respectively to create PEI₁₈₀₀-LinA5-PEG_{0.3} and PEI₁₀ₖ-Lin_{A15}-PEG_{3.0.} Functionalized polymers were dialyzed against water, extracted in diethyl ether, and lyophilized. PEI₆₀₀-MA5 was combined with cholesterol (Cho) and PEG-OA through microfluidic mixing for size optimization.

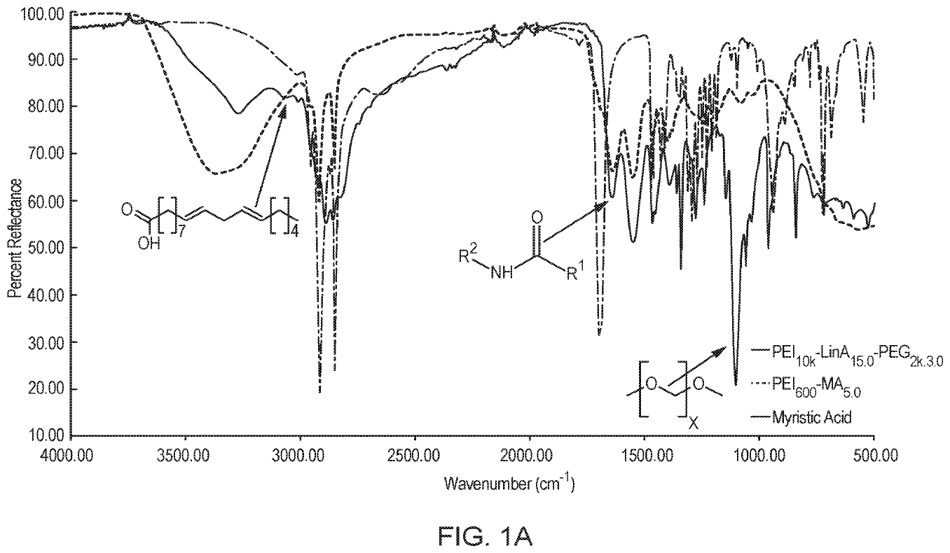

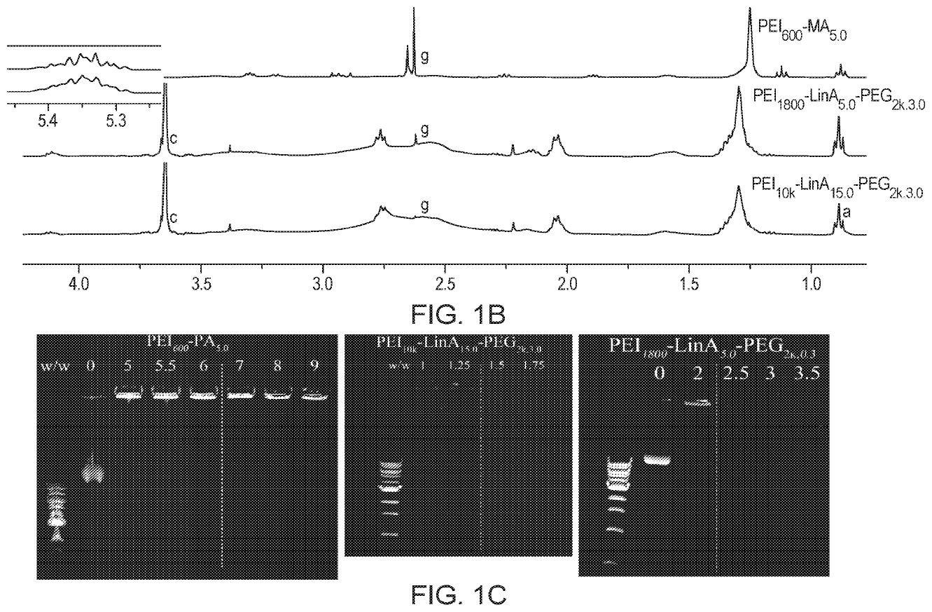

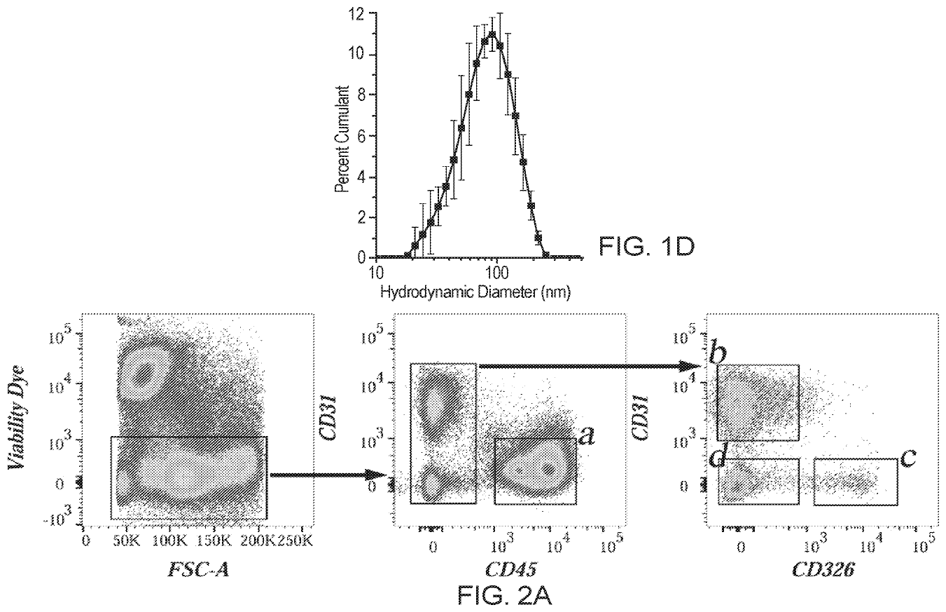

Attenuated total reflectance Fourier transform infrared (atr-FTIR) analysis confirmed successful amidation by appearance of an amide carbonyl v=1650 cm−1(s) in the conjugated polymers (FIG. 1, A). PEG, v=1100 cm−1(s; C—O), and the sp2 carbon bond of linoleic acid v=3050 cm−1(s; C═C) were observed in the PEI₁₀ₖ-Lin_{A15}-PEG3.0 spectrum (FIG. 1A) as well as by 1H NMR in CDC13 (FIG. 1B). Table 1 shows calculated DoC for PEI₆₀₀, PEI₁₈₀₀, and PEI₁₀ₖ. Conjugation is close to theoretical ratios for lower ratios used during PEI600 conjugation but begin to drift when using higher molecular weights.

TABLE 1
DoC for fatty acid and PEG2k conjugated
PEI determined by ¹H NMR

| | Fatty Acid | PEG₂ₖ |
|---|---|---|
| PEI₆₀₀-MA5 | 4.5 ± 0.1 | |
| PEI₁₈₀₀-LinA5-PEG_{0.3} | 6.5 ± 0.8 | 0.39 ± 0.03 |
| PEI₁₀ₖ-LinA₁₅-PEG_{3.0} | 20.7 ± 2 | 1.95 ± 0.15 |

Gel electrophoresis was used to determine the onset of stabilization. The onset of stabilization was taken to be the w/w which fully restricted DNA migration (FIG. 1C). Size quantifications for PEI₆₀₀-MAS, PEI1800-LinA5-PEG_{0.3}, and PEI₁₀ₖ-Lin_{A15}-PEG_{3.0} were done at w/w=21, 25, and 15 respectively in normal glucose and show sizes within the useful range for in-vivo application (Table 2).^{[36]} FIG. 1D shows monodisperse characteristics for the hydrodynamic diameter distribution of PEI₁₀ₖ-LinA₁₅-PEG_{3.0} polyplexes. The size optimized formulation of PEI₆₀₀-MA5:PEG-OA:Cho was a mass ratio of 100:11.1:11.1.

TABLE 2
Hydrodynamic sizes and zeta potentials
of colloids in normal glucose.

| | | Z-average | Zeta Potential |
|---|---|---|---|
| | w/w | (d · nm) | (mV) |
| PEI₆₀₀-MA5/PEG-OA/Cho | 21 | 123 ± 49 | 24.0 ± 5.1 |
| PEI₁₈₀₀-LinA5-PEG_{0.3} | 25 | 142 ± 66 | 22.2 ± 5.4 |
| PEI₁₀ₖ-LinA₁₅-PEG_{3.0} | 15 | 107 ± 56 | 23.7 ± 7.4 |

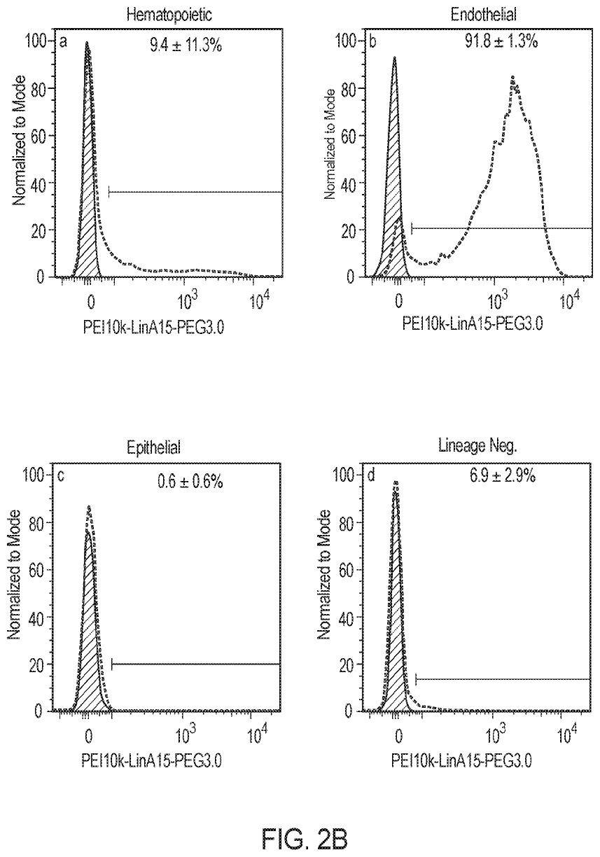

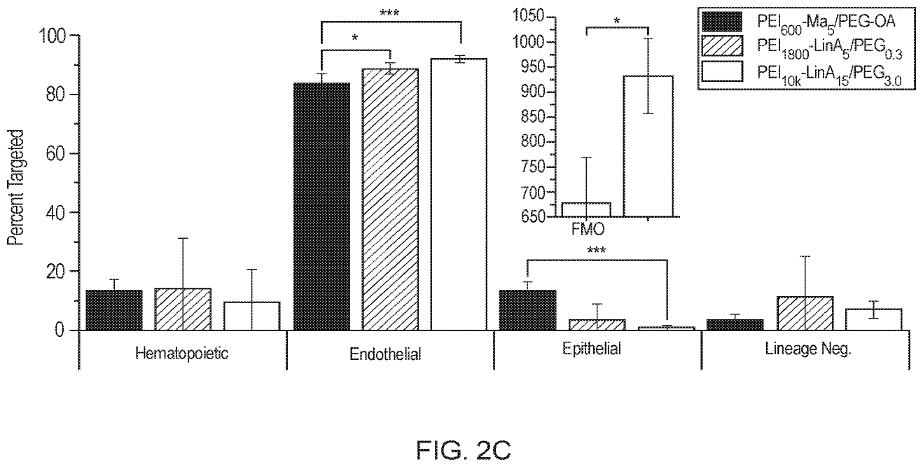

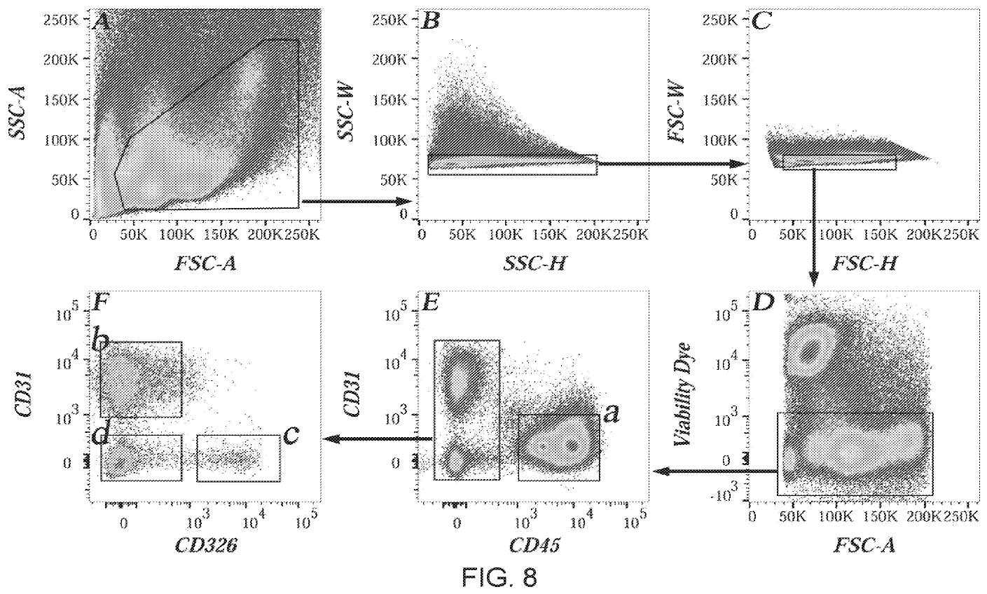

3.2 In-Vivo Targeting: For the investigation of targeting efficiency, functionalized PEI was mixed with 40 μg of purified plasmid DNA at mass ratios (w/w) dependent upon the onset of stabilization as quantified by gel electrophoresis and diluted in normal glucose. Targeting efficiency of DyLight 650 labeled nanopartiles was determined 24 hours post tail vein injection in healthy, adult male, wild type C57BL6/J mice by flow cytometry. Cell populations examined were gated as live singlet CD45+ CD31− (hematopoietic), CD31+ CD326− CD45− (endothelial), CD326+ CD31− CD45− (epithelial), and CD45− CD31− CD326− (lineage negative, cell population mostly containing fibroblasts and pericytes). FIG. 2A shows a representation of the gated populations with a full gating strategy presented in FIG. 8. FIG. 2B shows the fluorescent histogram for PEI10k-LinA15-PEG3.0 against the fluorescence minus one (FMO) control. A comparison of targeting efficiencies (n=3) is presented in FIG. 2C. Stabilized eGFP RNA complexed with PEI10k-LinA15-PEG3.0 was delivered intravenously in normal glucose; the median fluorescent intensity (MFI) from endothelial cells isolated 24 hours post injection was quantified by flow cytometry and was found to be significantly higher than control mice. (p<0.05, n=5) (FIG. 2C inset).

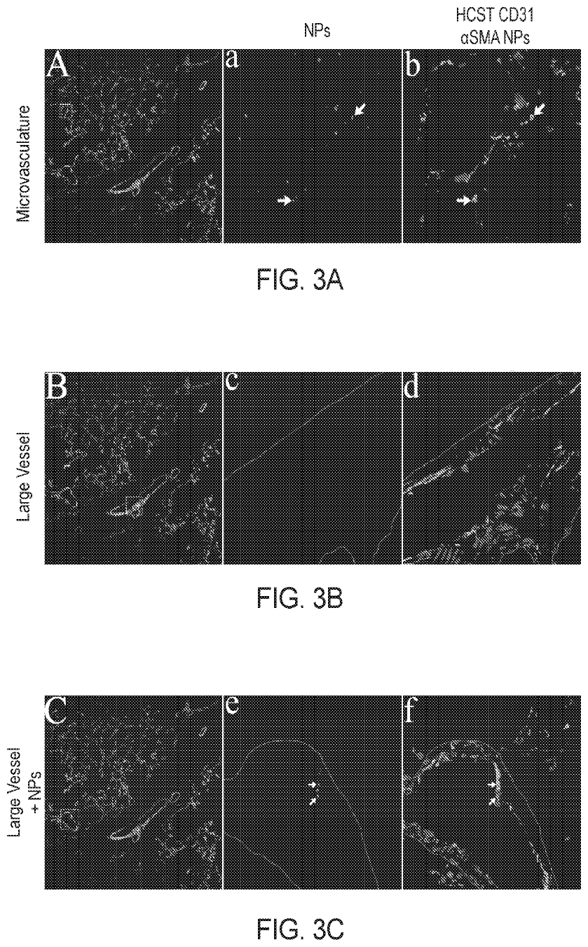

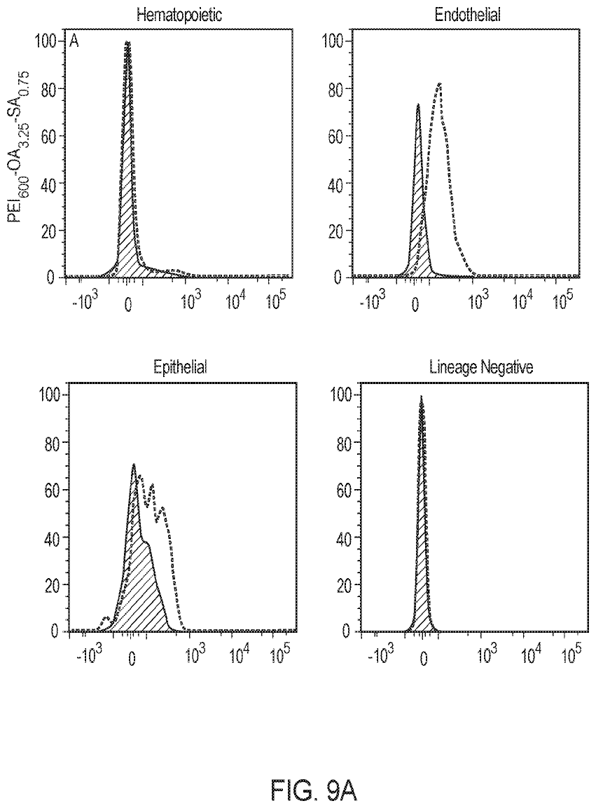

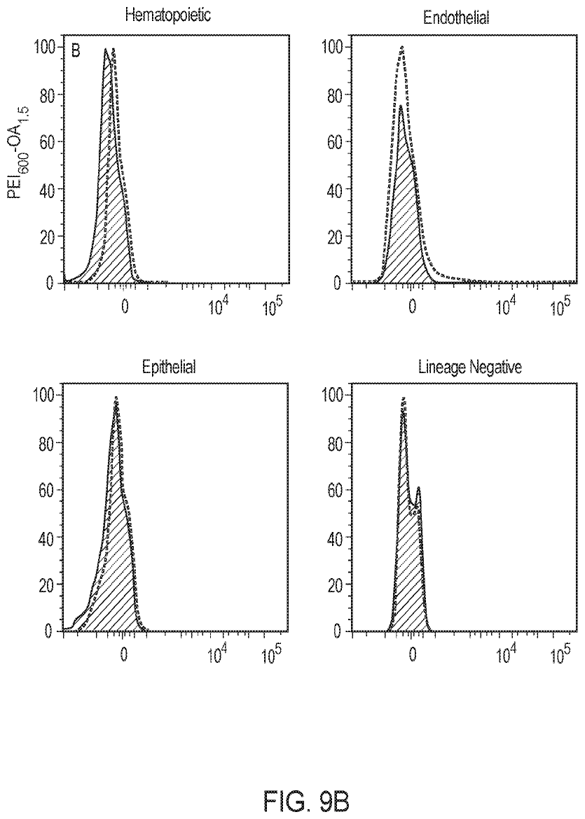

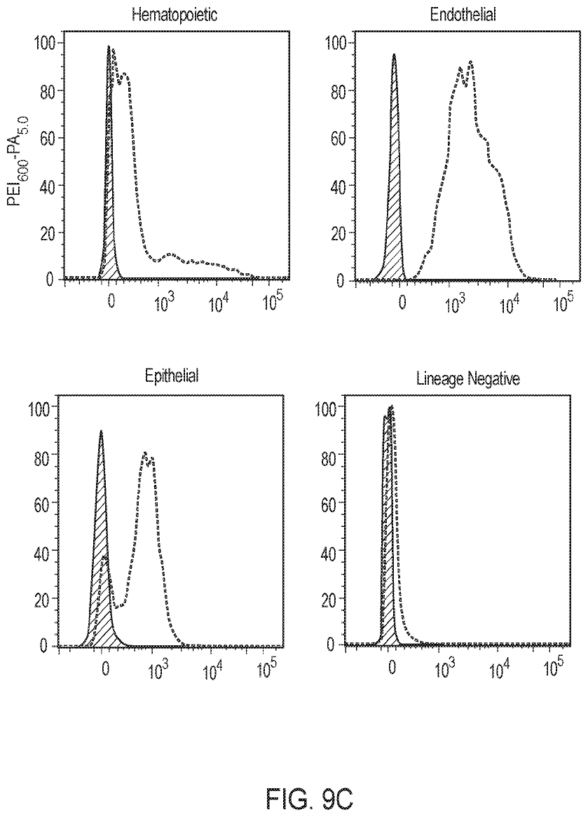

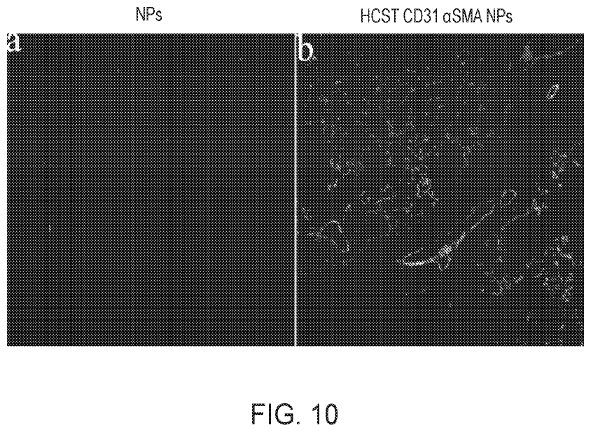

3.3 Immunofluorescence: The distribution of DyLight 650 tagged PEI10k-LinA15-PEG3.0 nanoparticles in the lung tissue was investigated using 10 μm frozen lung sections harvested from healthy adult male, wild type C57BL6/J mice 24 hours post tail vein injection. Sections were stained with Hoechst 33342 (nuclear stain), platelet endothelial cell adhesion molecule (PECAM1, CD31), and alpha smooth muscle actin (αSMA) for visualization of microvasculature and large vessels. Confocal images of stained sections show that nanoparticles (NPs) were highly disseminated throughout the pulmonary microvasculature (FIG. 3A, FIGS. 9A-9C) as shown by co-localization of DyLight with PECAM1 (FIG. 3A panel b). NPs within the lumen of larger vessels were sparse (FIG. 3B panel c, FIG. 3C panel e). This is likely a result of hemodynamic differences between large vessels and capillary beds. FIG. 3C panel f shows NPs found within the lumen of large vessels colocalized with PECAM1

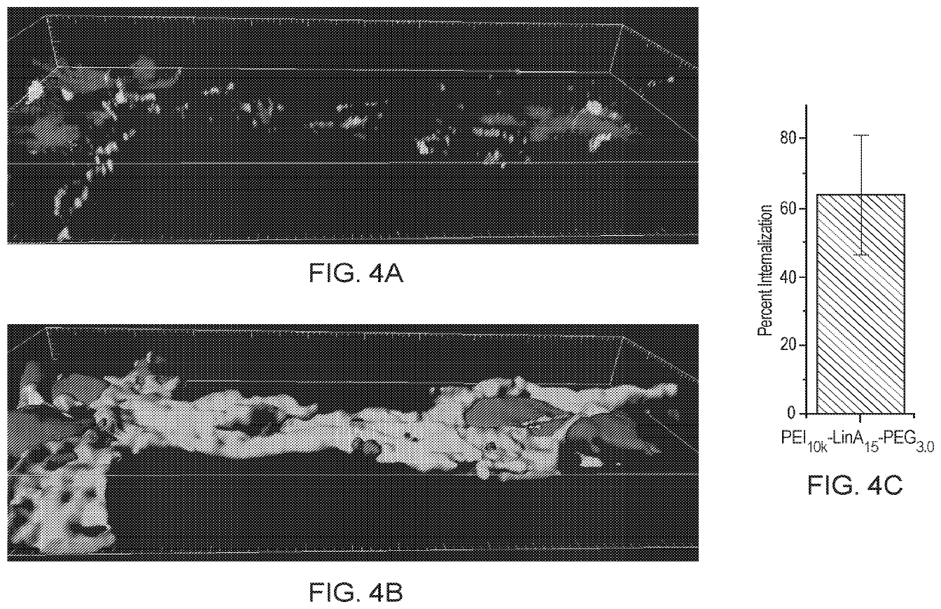

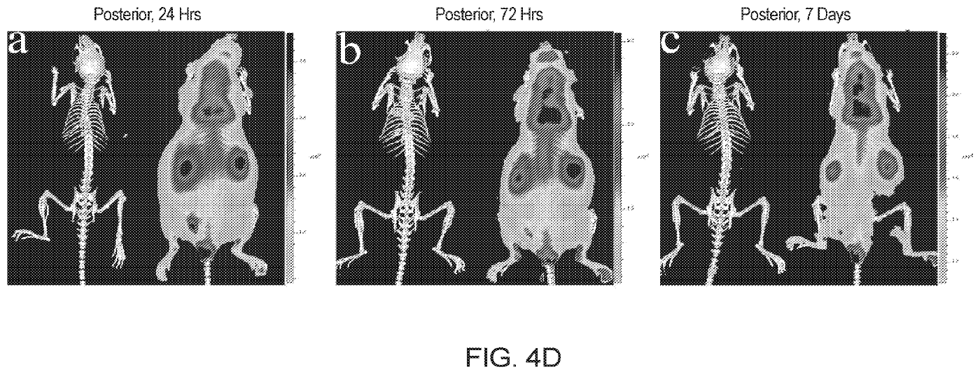

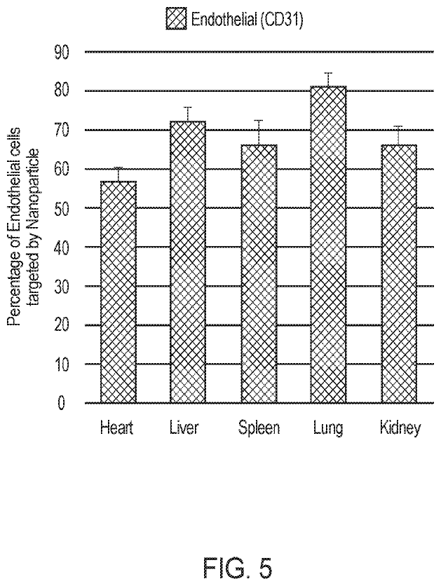

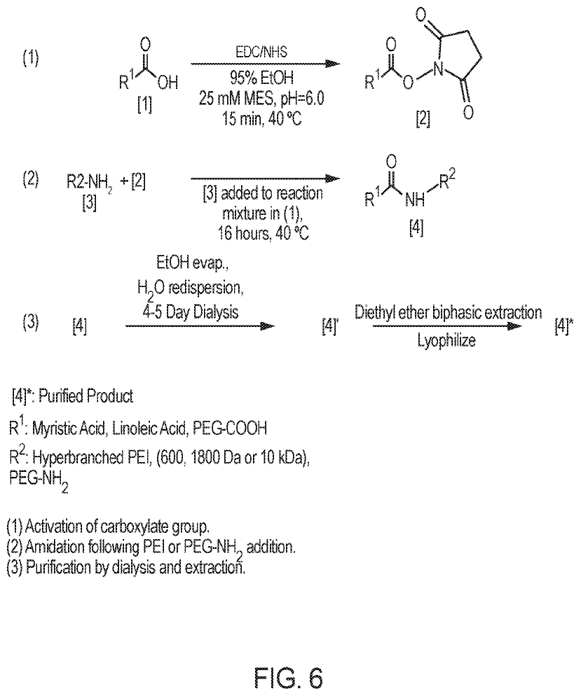

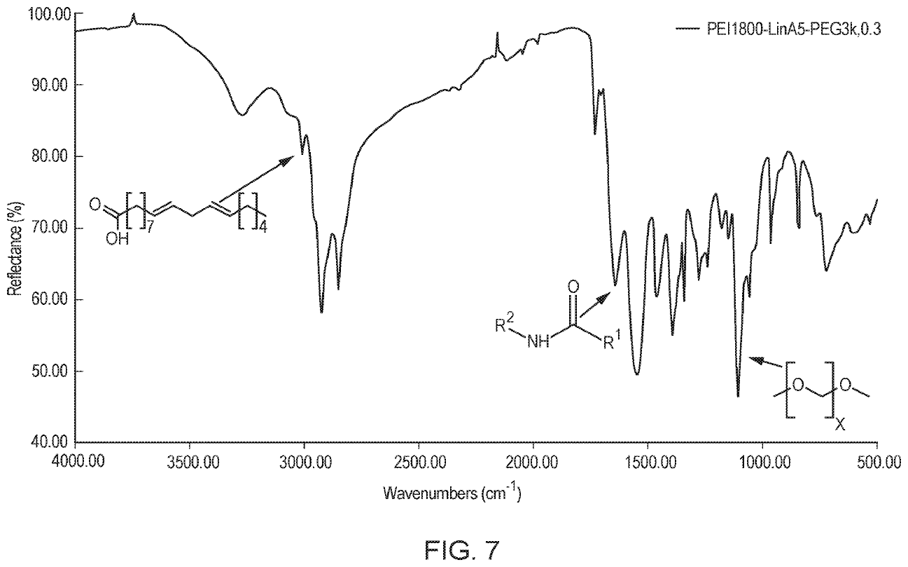

3.4 Biodistribution: Richardson-Lucy deconvolution was performed on a Z-stack image of lung microvasculature. FIG. 4A, shows a 3D maximum intensity projection of a deconvoluted Z-stack showing Hoechst nuclear staining (blue), PECAM1 (green), PEI10k-LinA15-PEG3.0 (red). This maximum intensity plot was subsequently used for the automated surface plot generation in Imaris and used for determining the percentage of nanoparticle internalization (FIG. 4B). The internalization, as calculated based off nanoparticle fluorescence within the PECAM1 surface stain, was found to be 63.8±17.6% (FIG. 4C). For investigation of possible targeting in other organ systems, live in-vivo imaging was completed using an IVIS SpectrumCT. Mice were given DyLight 650 conjugated PEI10k-LinA15-PEG3.0 complexed with 40 μg of plasmid DNA in normal glucose injected as a 200 μl bolus through the tail vein. An uninjected control mouse (left) was imaged simultaneously alongside an injected mouse (right) at each time point (FIG. 4D). Acquisition shows maximal accumulation in regions near the lung and kidneys with a signal that was stable for at least 7 days.

### Discussion

In this study, Applicant generated three novel formulations of PEI-based polyplexes that target pulmonary microvascular endothelium with high specificity. Low molecular weight hyperbranched PEI was easily functionalized with biological fatty acids and PEG. The conjugation of fatty acids onto low molecular weight PEI was completed by amidation using EDC/NHS coupling. For PEI600, conjugation by 1H NMR analysis was found to closely match the theoretical degree of conjugation. However, slight deviations from the theoretical degree of conjugation were observed for PEI1800 and PEI10k.

PEI₁₈₀₀-LinA5-PEG_{0.3} was found to significantly target a larger population of endothelial cells compared to PEI₆₀₀-MA5/PEG-OA/Cho (p<0.05) but juxtaposition of targeted hematopoietic, epithelial, and lineage negative populations revealed no significant differences. PEI₁₀ₖ-LinA₁₅-PEG_{3.0} was found to significantly target a greater population of endothelial cells compared to PEI₆₀₀-MA5/PEG-OA/Cho (p<0.001) and a smaller population of epithelial cells (p<0.001); hematopoietic and lineage negative populations remained not significantly different. This increase in endothelial targeting is likely a result of improved intravascular stabilization with increased PEG grafting, leading to improved dissemination throughout the lung microvasculature, as initial colloid size and surface potential for the three formulations do not present any significant differences at the mass ratios used.^{[34, 37, 38]} Furthermore, fluorescent quantification by flow cytometry on endothelial cells isolated from mice 24 hours post intravenous injection with 30 μg of stabilized eGFP RNA complexed with PEI₁₀ₖ-LinA₁₅-PEG_{3.0} showed a significant increase in MFI indicating the ability for PEI₁₀ₖ-LinA₁₅-PEG_{3.0} to successfully deliver RNA for translation into active protein. High specificity is not a global trait of all PEI based cationic nanoparticles. Specificity is strongly dependent upon grafting density and type of fatty acid used as revealed by initial screening (FIG. 9) This variation was found to be dependent upon colloidal properties with initial, highly positive surface potential correlating with reduced targeting efficiency (Table 3).

TABLE 3
Size and Zeta Potential of selected colloids from initial screens

| | Z-average | Zeta Potential |
|---|---|---|
| | (d · nm) | (mV) |
| PEI₁₈₀₀-OA_{3.25}-SA_{0.75} | 113 ± 59 | 13.7 ± 8.16 |
| PEI₆₀₀-OA_{1.5} | 142 ± 78 | 45.5 ± 6.44 |
| PEI₆₀₀-MA₅ | 255 ± 90 | 14 ± 5 |

These three specific formulations targeted 85-95% of pulmonary endothelial cells showing a significantly higher targeting efficiency compared to PEI formulations previously reported in the literature. ^{[20]} However the mechanism behind such robust, non-affinity targeting is not fully understood.

Nanoparticle uptake is important for successful delivery. 3D deconvolution and surface reconstruction of PECAM1(+) endothelial cells indicated that a majority of PEI₁₀ₖ-LinA₁₅-PEG_{3.0} nanoparticles were within endothelial cells 24 hours post injection by internalization of measured fluorescence. While PEI₁₀ₖ-LinA₁₅-PEG_{3.0} nanoparticle uptake is observed, it presently remains unclear as to what is the dominating mechanism as nanoparticles are known to endocytose by a multitude of routes, with dependencies on size and surface chemistry, including clathrin/caveolar mediated endocytosis, phagocytosis, and macropinocytosis. ^{[39, 40]} Whole body biodistribution of DyLight 650 conjugated PEI₁₀ₖ-LinA₁₅-PEG_{3.0} in adult nude mice was examined using an IVIS SpectrumCT. Live imaging revealed whole body dissemination with concentration near the lungs and kidneys; relative fluorescence distribution appeared static and was observable for the entirety of the 7-day study. This result reflects known biodegradability and clearance properties of PEI based nanoparticles.^{[41]}

### CONCLUSION

In summary, Applicant has developed a nanoparticle system based off low molecular weight, hyperbranched PEI through a synthesis route that has allowed for a one pot, unique conjugation scheme using PEG and biological fatty acids under green conditions. (Green conditions generally refer to a synthesis route that may have lower environmental impact, based off the solvents used, for example, ethanol and other simple alcohols are considered to be more environmentally friendly than alternatives such as DMF, THF, or Dioxane.) Colloidal characterization has revealed a size and zeta potential near 120 (d.nm.) and +24 mV in normal glucose respectively with a targeting percentage of >85%. Without intending to be limited by theory, it is believed that this combination of size and zeta potential, derived from the specific formulations of the polymeric nanoparticles, which has allowed these colloidal systems to efficiently target and deliver nucleic acids for successful protein expression to the pulmonary microvascular network through charge based, passive targeting in an uninjured mouse model with a targeting efficiency of 91.8±1.3% of endothelial cells. Live imaging revealed whole body distribution with the kidneys as further possible targets.

Statistics: Values are reported as mean±16. Significance was calculated using an unpaired Welch's t-test assuming unequal variance.

### Preparation of Nnanoparticles

Methoxypolyethylene glycol amine Mn=2000 (PEGNH₂) is obtained from Nanocs. Polyethylenimine (Mn=600), Myristic Acid (MA) ≥99%, Oleic Acid (OA ≥99%), Cholesterol (BioReagent ≥99%), Ethanol (EtOH, 200p), HPLC grade water, 2-(N-morpholino)ethanesulfonic acid (MES) ≥99% and 3-(N-Morpholino)propanesulfonic acid (MOPS) is obtained through Sigma-Aldrich and used without further purification. 1-ethyl-3-(3-dimethylaminopropyl)carbodiimide hydrochloride (EDC), N-hydroxysuccinimide (NHS), DyLight 650 NHS Ester, and Spectrum™ Spectra/Por™ 3.5 kDa Slide-A-Lyzer™ are obtained through ThermoFisher Scientific. Diethyl ether (anhydrous, BHT stabilized), and 20 kDa MWCO dialysis tubing were obtained through Fisher Scientific.

Functionalization of PEI with biological fatty acids and PEG is completed through amidation using EDC/NHS mediated coupling in 95% ethanol buffered with 25 mM MES, pH=6. Carboxylate groups are reacted by EDC/NHS for 15 minutes at 40° C. PEI or PEGNH₂ is quickly added following carboxylate group activation and is allowed to react overnight at 40° C. to create PEI600-MA5 or PEG-OA. Ethanol is removed by rotary evaporation, the polymer resuspended in water, and dialyzed against water using a 20 kDa membrane for 4-5 days. Colloids are then extracted twice in diethyl ether and lyophilized. Cholesterol is dissolved in ethanol. Lyophilized polymers are suspended in 10 mM MOPS, pH=7.4. PEI₆₀₀-MA5 is stabilized with cholesterol and PEG-OA through solvent diffusion and microfluidic mixing at a mass ratio of 85:15:10, PEI:Cholesterol:PEG. PEI₆₀₀-MA5 is conjugated with DyLight 650 overnight at room temperature in 10 mM MOPS. Residual ethanol is removed by dialysis against an isotonic dextran solution using a 3.5 kDa Slide-A-Lyzer™. Intravenous injections are performed using colloids mixed with plasmids at a mass ratio (w/w) of 24 in normal glucose. 5 μg plasmids in 20 μl is used for intravenous injections in neonatal mice. Infrared spectroscopy is run on a Nicolet attenuated total reflection Fourier transform infrared (atr-FTIR) spectrometer outfitted with a diamond crystal. Hydrodynamic size and zeta potential are measured on a Malvern Zetasizer Nano ZS in normal glucose.

### Nanoparticle Mediated Delivery of STAT3 Restores Endothelial Proliferation and Stimulates Angiogenesis in S52F-Foxf1 Mutant Lungs

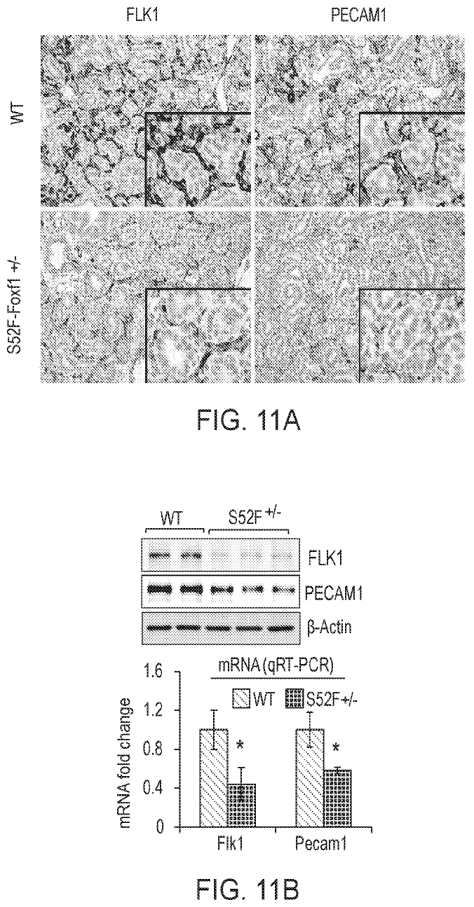

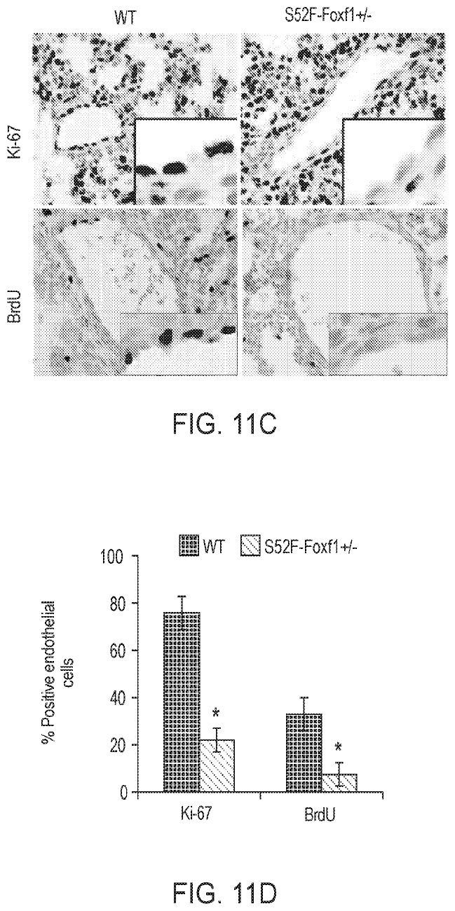

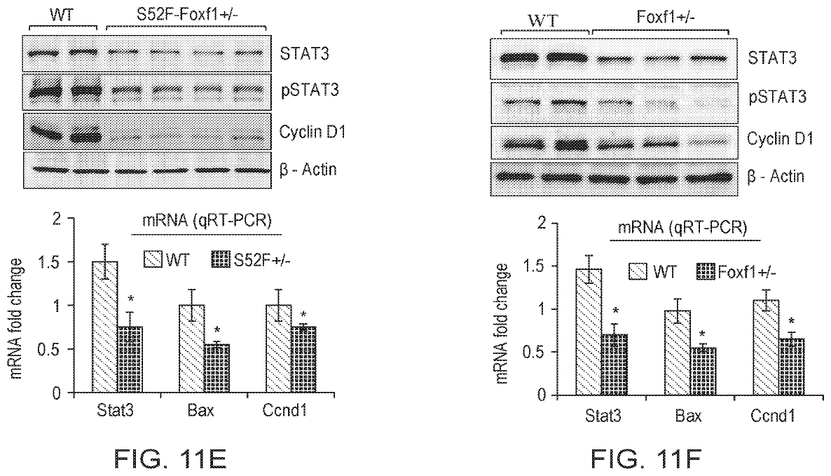

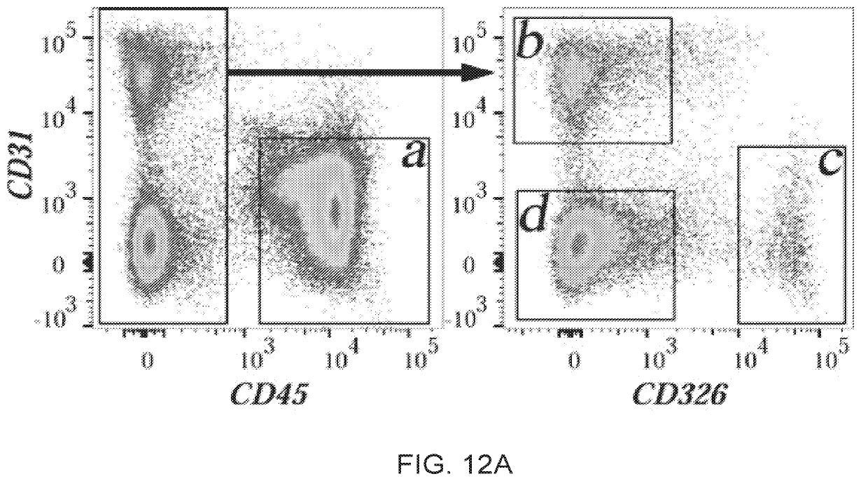

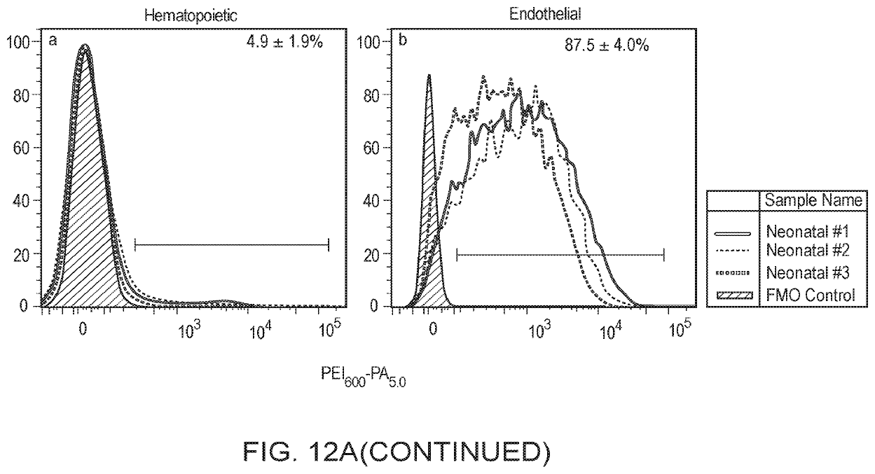

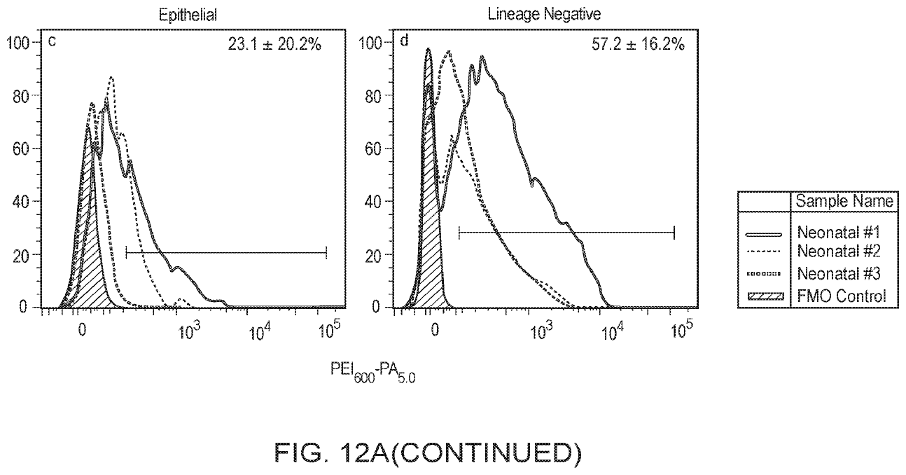

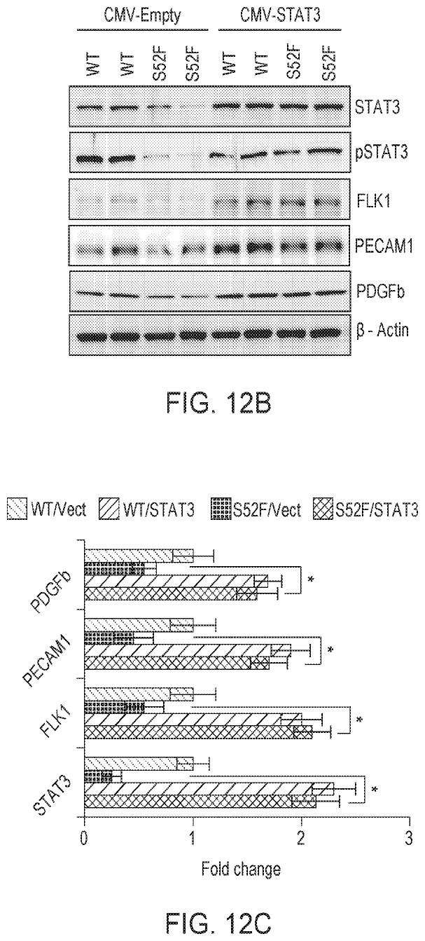

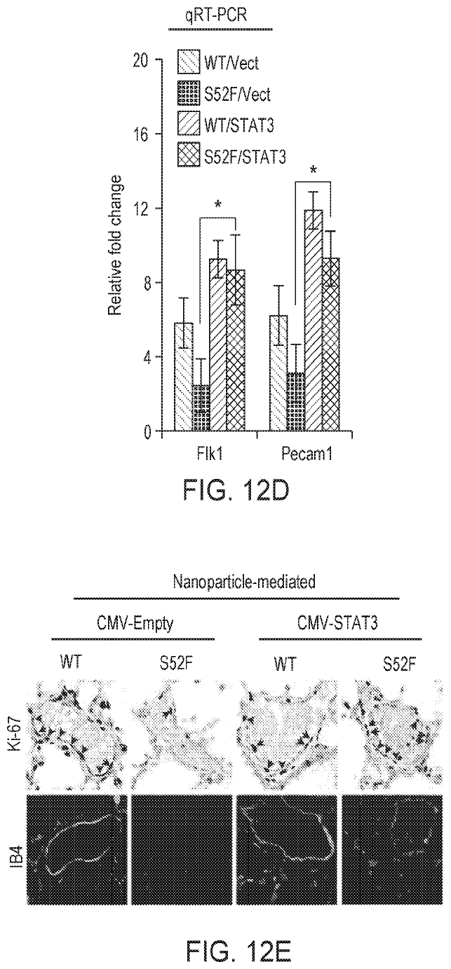

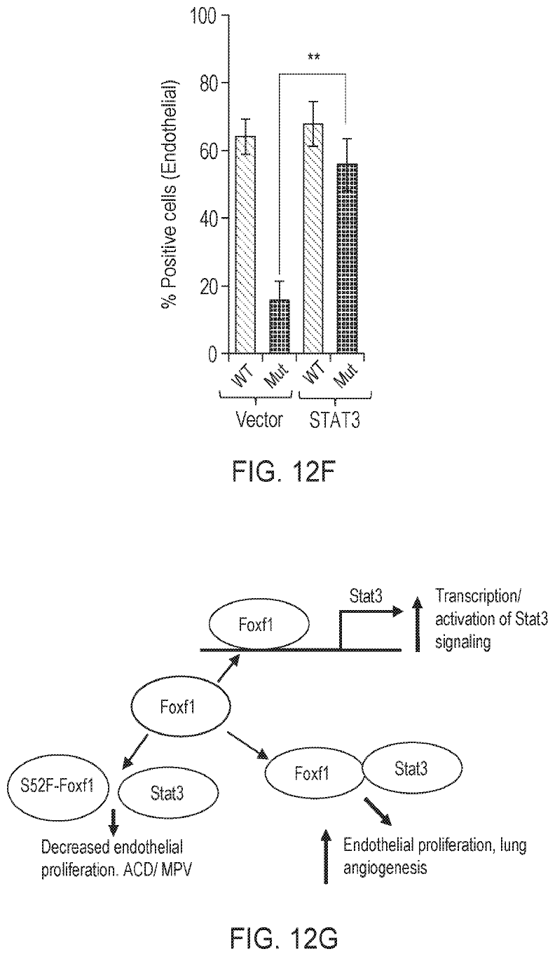

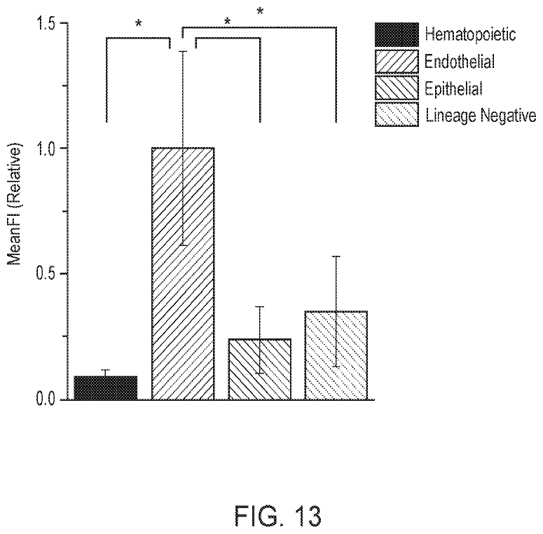

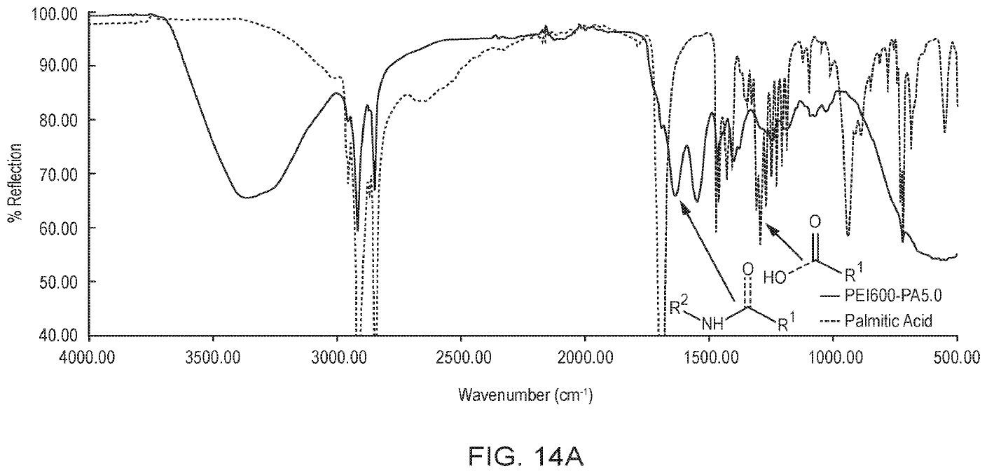

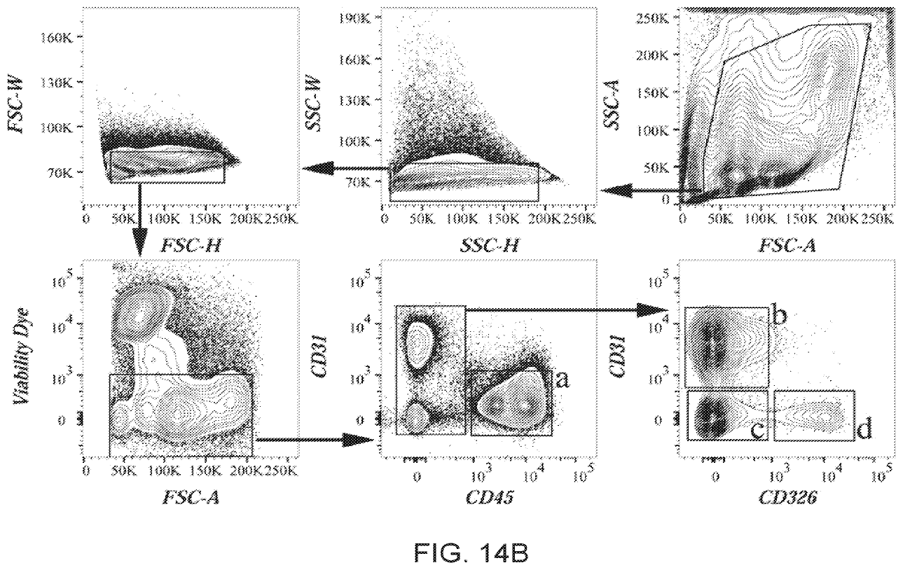

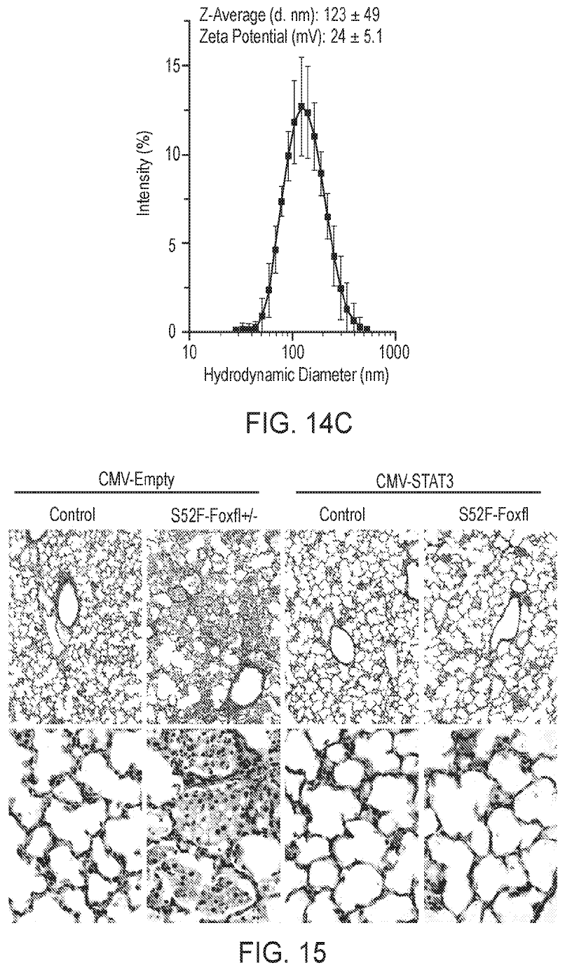

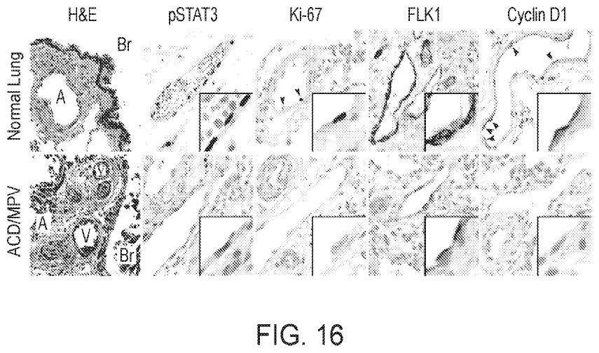

STAT3 stimulates proliferation of endothelial cells in vitro and in vivo.^{33, 38} Since STAT3 was reduced in Foxfl-deficient mice (FIG. 11F) and ACDMPV lungs (FIG. 16) Applicant tested whether restoring STAT3 signaling in S52F Foxf1+/− newborns would enhance pulmonary endothelial proliferation and angiogenesis. To deliver Stat3 cDNA, Applicant used PEI nanoparticles that were capable of delivering gene constructs and shRNAs in vivo.³⁹ To improve the efficiency of the in vivo targeting, Applicant used the EDC/NHS conjugation strategy to create a novel formulation of PEI nanoparticles, PEI 600-MA5.0, which was stabilized with cholesterol and PEG2K-OA (FIGS. 14B -14C). Fluorescently labeled PEI 600-MA5.0 nanoparticles were used to deliver a single dose of Stat3 cDNA into the facial vein of newborn pups. After gene delivery, nanoparticles were detected by FACS analysis in 88% of lung endothelial and 57% of mesenchymal cells (FIG. 12A, and FIG. 13). Nanoparticles were ineffective in targeting hematopoietic and epithelial cells in the lung tissue (FIG. 12A). Stat3 cDNA increased total STAT3 protein and STAT3 phosphorylation in S52F Foxf1 lungs as shown by Western blot (FIGS. 12B-12C). After Stat3 cDNA delivery, lung angiogenesis was improved in S52F Foxf1 mice as evidenced by increased mRNA and protein levels of endothelial markers PECAM1, FLK1, and PDGFb (FIGS. 12B-12D) enhanced ability of endothelial cells to bind isolectin B4 (FIG. 12E) and increased numbers of Ki-67-positive endothelial cells in S52F Foxf1+/− lungs (FIGS. 12E and 12F). Finally, Stat3 cDNA decreased lung inflammation and improved alveogenesis in S52F Foxf1+/− mice (FIG. 15). Altogether, the data indicate that STAT3 is a key target of FOXF1 regulating angiogenesis in ACDMPV.

### Exemplary Formulation

An exemplary composition may be: 40 μg plasmid DNA mixed with 960 μg PEI₆₀₀-MA5/PEG-OA/Cho in normal glucose buffered to pH 7.4 by 10 or 25 mM of a biological buffer. (Buffers may include, for example, MOPS (3-(N-morpholino)propanesulfonic acid) or HEPES (4-(2-hydroxyethyl)-1-piperazineethanesulfonic acid))

### REFERENCES

[1] R. H. Steinhorn, Pediatric critical care medicine: a journal of the Society of Critical Care Medicine and the World Federation of Pediatric Intensive and Critical Care Societies. 2010, 11(2 Suppl), S79.

[2] N. W. Morrell, S. Adnot, S. L. Archer, J. Dupuis, P. L. Jones, M. R. MacLean, I. F. McMurtry, K. R. Stenmark, P. A. Thistlethwaite, N. Weissmann, J. Am. Coll. Cardiol. 2009, 54(1 Supplement), S31.

[3] J. Alameh, A. Bachiri, L. Devisme, P. Truffert, T. Rakza, Y. Riou, S. Manouvrier, P. Lequien, L. Storme, Eur. J. Pediatr. 2002, 161(5), 262-266.

[4] H. Kool, D. Mous, D. Tibboel, A. Klein, R. J. Rottier, Birth Defects Research Part C: Embryo Today: Reviews. 2014, 102(4), 343-358.

[5] P. M. Mourani, S. H. Abman, Curr. Opin. Pediatr. 2013, 25(3), 329-337.

[6] P. M. Mourani, M. K. Sontag, A. Younoszai, J. I. Miller, J. P. Kinsella, C. D. Baker, B. B. Poindexter, D. A. Ingram, S. H. Abman, American Journal of Respiratory and Critical Care Medicine. 2015, 191(1), 87-95.

[7] A. J. Bhatt, G. S. Pryhuber, H. Huyck, R. H. Watkins, L. A. Metlay, W. M. Maniscalco, American Journal of Respiratory and Critical Care Medicine. 2001, 164(10), 1971-1980.

[8] L. Farkas, D. Farkas, K. Ask, A. Miler, J. Gauldie, P. Margetts, M Inman, M. Kolb, J. Clin. Invest. 2009, 119(5), 1298-1311.

[9] Z. Cao, R. Lis, M. Ginsberg, D. Chavez, K. Shido, S. Y. Rabbany, G. Fong, T. P. Sakmar, S. Rafii, B. Ding, Nat. Med. 2016.

[10] D. A. Muruve, M. J. Barnes, I. E. Stillman, T. A. Libermann, Hum. Gene Ther. 1999, 10(6), 965-976.

[11] Y. Zhang, N. Chirmule, G. Gao, R. Qian, M. Croyle, B. Joshi, J. Tazelaar, J. M. Wilson, Molecular Therapy. 2001, 3(5), 697.

[12] D. A. Muruve, M. J. Cotter, A. K. Zaiss, L. R. White, Q. Liu, T. Chan, S. A. Clark, P. J. Ross, R. A. Meulenbroek, G. M. Maelandsmo, R. J. Parks, J. Virol. 2004, 78(11), 5966-5972.

[13] D. A. Muruve, Hum. Gene Ther. 2004, 15(12), 1157-1166.

[14] S. Nayak, R. W. Herzog, Gene Ther. 2010, 17(3), 295-304.

[15] W. T. Godbey, K. K. Wu, A. G. Mikos, J. Biomed. Mater. Res. 1999, 45(3), 268-275.

[16] W. T. Godbey, K. K. Wu, A. G. Mikos, J. Controlled Release. 1999, 60(2), 149-160.

[17] U. Lungwitz, M. Breunig, T. Blunk, A. Gopferich, Eur. J. Pharm. Biopharm. 2005, 60(2), 247-266.

[18] H. C. Kang, H. Kang, Y. H. Bae, Biomaterials. 2011, 32(4), 1193-1203.

[19] A. Schroeder, J. E. Dahlman, G. Sahay, K. T. Love, S. Jiang, A. A. Eltoukhy, C. G. Levins, Y. Wang, D. G. Anderson, J. Controlled Release. 2012, 160(2), 172-176.

[20] O. F. Khan, E. W. Zaia, S. Jhunjhunwala, W. Xue, W. Cai, D. S. Yun, C. M. Barnes, J. E. Dahlman, Y. Dong, J. M. Pelet, Nano Letters. 2015, 15(5), 3008-3016.

[21] J. Dai, S. Zou, Y. Pei, D. Cheng, H. Ai, X. Shuai, Biomaterials. 2011, 32(6), 1694-1705.

[22] M. Zheng, Y. Zhong, F. Meng, R. Peng, Z. Zhong, Molecular pharmaceutics. 2011, 8(6), 2434-2443.

[23] G. Navarro, S. Essex, R. R. Sawant, S. Biswas, D. Nagesha, S. Sridhar, C. T. de ILarduya, V. P. Torchilin, Nanomedicine: Nanotechnology, Biology and Medicine. 2014, 10(2), 411-419.

[24] J. Li, D. Cheng, T. Yin, W. Chen, Y. Lin, J. Chen, R. Li, X. Shuai, Nanoscale. 2014, 6(3), 1732-1740.

[25] A. Masotti, F. Moretti, F. Mancini, G. Russo, N. Di Lauro, P. Checchia, C. Marianecci, M. Carafa, E. Santucci, G. Ortaggi, Bioorg. Med. Chem. 2007, 15(3), 1504-1515.

[26] G. Guo, L. Zhou, Z. Chen, W. Chi, X. Yang, W. Wang, B. Zhang, Int. J. Pharm. 2013, 450(1-2), 44-52.

[27] L. Liu, M. Zheng, T. Renette, T. Kissel, Bioconjug. Chem. 2012, 23(6), 1211-1220.

[28] A. Zintchenko, A. Philipp, A. Dehshahri, E. Wagner, Bioconjug. Chem. 2008, 19(7), 1448-1455.

[29] R. M. Schiffelers, A. Ansari, J. Xu, Q. Zhou, Q. Tang, G. Storm, G. Molema, P. Y. Lu, P. V. Scaria, M. C. Woodle, Nucleic Acids Res. 2004, 32(19), e149.

[30] M. L. Forrest, J. T. Koerber, D. W. Pack, Bioconjug. Chem. 2003, 14(5), 934-940.

[31] P. Y. Teo, C. Yang, J. L. Hedrick, A. C. Engler, D. J. Coady, S. Ghaem-Maghami, A. J. T. George, Y. Y. Yang, Biomaterials. 2013, 34(32), 7971-7979.

[32] G. T. Noble, J. F. Stefanick, J. D. Ashley, T. Kiziltepe, B. Bilgicer, Trends Biotechnol. 2014, 32(1), 32-45.

[33] J. E. Dahlman, C. Barnes, 0. F. Khan, A. Thiriot, S. Jhunjunwala, T. E. Shaw, Y. Xing, H. B. Sager, G. Sahay, L. Speciner, Nature Nanotechnology. 2014, 9(8), 648-655.

[34] R. Gref, M. Luck, P. Quellec, M. Marchand, E. Dellacherie, S. Harnisch, T. Blunk, R. H. Muller, Colloid Surf. B-Biointerfaces. 2000, 18(3-4), 301-313.

[35] C. Fang, B. Shi, Y. Y. Pei, M. H. Hong, J. Wu, H. Z. Chen, Eur. J. Pharm. Sci. 2006, 27(1), 27-36.

[36] A. E. Nel, L. Madler, D. Velegol, T. Xia, E. M. Hoek, P. Somasundaran, F. Klaessig, V. Castranova, M. Thompson, Nature Materials. 2009, 8(7), 543-557.

[37] S. M. Moghimi, A. C. Hunter, J. C. Murray, Pharmacol. Rev. 2001, 53(2), 283-318.

[38] B. Romberg, W. E. Hennink, G. Storm, Pharm. Res. 2008, 25(1), 55-71.

[39] W. Jiang, B. Y. S. Kim, J. T. Rutka, W. C. W. Chan, Nat. Nanotechnol. 2008, 3(3), 145-150.

[40] N. Oh, J. Park, International journal of nanomedicine. 2014, 9(Suppl 1), 51.

[41] Y. Wen, S. Pan, X. Luo, X. Zhang, W. Zhang, M. Feng, Bioconjug. Chem. 2009, 20(2), 322-332.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A composition for delivery of a therapeutic agent, wherein said composition comprises a polyethylenimine (PEI) conjugated to a fatty acid (FA) to form a PEI-FA conjugate, wherein said PEI-FA conjugate aggregates to form a micelle.

2. The composition of claim 1, wherein said PEI-FA conjugate is conjugated to a carboxylate-terminated polyethylene glycol (PEG) to form a PEI-FA-PEG conjugate, wherein said PEI-FA-PEG conjugate aggregates to form a micelle.

3. The composition of claim 1, wherein said PEI has an Mn (number average molecular weight) of from about 600 Da to about 10 kDa, or about 1000 Da to about 2500 Da, or about 1200 Da to about 1800 Da, wherein Mn is defined as (when n=1):
$$\frac{{\sum }_{i = 1}^{p} {Mn}_{i}^{n} * {N}_{i}}{{\sum }_{i = 1}^{p} {Mn}_{i}^{n - 1} * {N}_{i}}$$
where the molecular weight distribution is quantized into (p) fractions, (Ni) and (Mnᵢ) are the number of molecules in the i^{th} fraction and molecular weight in the i^{th} fraction respectively.

4. The composition of claim 1, wherein said polyethylenimine (PEI) is a branched polyethylenimine (PEI).

5. The composition of claim 1, wherein said micelle is a cationic micelle.

6. The composition of claim 1, wherein said PEG has an Mn (number average molecular weight) of from about 2 kDa to about 5 kDa.

7. The composition of claim 1, wherein said PEI has an Mn (number average molecular weight) of about 600 Da to about 10 kDa.

8. The composition of claim 1, wherein said PEI-FA ratio is from about 3 to about 30, or wherein said PEG-FA ratio is from about 1 to about 2.

9. The composition of claim 1, wherein said micelle has a molar conjugation ratio (grafting density) of about 3 to about 5 moles of fatty acids per mole of PEI₆₀₀.

10. The composition of claim 1, wherein said micelle has a molar conjugation ratio (grafting density) of about 3 to about 8 moles of fatty acids per mole of PEI₁₈₀₀

11. The composition of claim 1, wherein said micelle has a molar conjugation ratio (grafting density) of about 3 to about 30 moles of fatty acids per mole of PEI₁₀ₖ

12. The composition of claim 1, wherein said micelle has a size of from about 80 nm to about 200 nm, or about 100 nm to about 150 nm, as quantified by Dynamic Light Scattering (DLS).

13. The composition of claim 1, wherein said micelle has a Zeta (Surface) Potential of from about 5 mV to about 34 mV, or about 20 mV to about 30 mV as quantified by Dynamic Light Scattering (DLS).

14. The cationic nanoparticle of claim 1 wherein said fatty acid is a biological fatty acid.

15. The cationic nanoparticle of claim 1 wherein said fatty acid is selected from any saturated or unsaturated fatty acid with a tail length of 12-16 carbons.

16. The composition of claim 1, wherein said micelle further comprises cholesterol.

17. The composition of claim 1, further comprising a therapeutic agent selected from a hydrophobic peptide, a hydrophobic small molecule, or a nucleic acid, wherein said micelle incorporates or encapsulates said therapeutic agent for delivery to an individual in need thereof.

18. The composition of claim 1, wherein said therapeutic agent is a nucleic acid selected from DNA and RNA.

19. The composition of claim 1 wherein said therapeutic agent is a nucleic acid selected from a pro-angiogenic or anti-angiogenic gene.

20. (canceled)

21. (canceled)

22. The composition of claim 1, wherein said zeta potential of said micelle is from about 5 to about 35 mV, or about 20 to about 30 mV.

23. The composition of claim 1, wherein composition is proved in a solution having a pH between about 7.3 to about 7.5 as measured by electrochemical potential.

24. The composition of claim 1, wherein said composition is provided in normal glucose buffered to physiological pH.

25. The composition of claim 1, further comprising glucose or trehalose.

26. A method of targeting a therapeutic agent to an individual having an endothelial-based disease comprising administering the composition of claim 1 to said individual.

27. The method of claim 26, wherein said endothelial-based disease is a pulmonary vascular disease selected from pulmonary hypertension, alveolar capillary dysplasia, arterial malformation, venous malformation, lymphatic malformation, bronchopulmonary dysplasia, pulmonary fibrosis, cystic obstructive pulmonary disease (COPD), interstitial lung disease, emphysema, a cancer, or combinations thereof.

28. The method of claim 26, wherein said administering step comprises intravenous administration.

29. The method of claim 26, wherein said nucleic acid is STAT3, FoxF1, a pro-angiogenic gene, an anti-angiogenic gene, or combinations thereof.
